(19) United States
(12) Patent Application Publication
(10) Pub. No.: US 2011/0276071 A1
(43) Pub. Date: Nov. 10, 2011

(54) Vasospasm-reducing aneurysm clip

(76) Inventors: Robert A. Connor, Minneapolis, MN (US); Muhammad Tariq Janjua, Inver Grove Heights, MN (US)

(21) Appl. No.: 13/066,155

(22) Filed: Apr. 19, 2011

Related U.S. Application Data
(60) Provisional application No. 61/395,142, filed on May 8, 2010.

(51) Int. Cl. A61B 17/08

(52) U.S. Cl. 606/158

(57) ABSTRACT

This invention is an implantable device that is attached to the exterior of an aneurysm, or to the exterior of some other portion of a blood vessel wall, wherein this device elutes, or otherwise emits, an emitted substance to prevent or treat vasospasm. This device may be embodied as a magnesium-eluting aneurysm clip. A magnesium-eluting aneurysm clip not only stops blood from escaping out of the aneurysm, but it also helps to prevent vasospasm from any blood that has already escaped.

### CROSS-REFERENCE TO RELATED APPLICATIONS

This patent application claims the priority benefit of U.S. Provisional Patent Application No. 61395142 entitled “Vasospasm-Reducing Aneurysm Clip” filed on May 8, 2010 by Tariq Janjua and Robert Connor.

### FEDERALLY SPONSORED RESEARCH

Not Applicable

### SEQUENCE LISTING OR PROGRAM

Not Applicable

### BACKGROUND

### Field of Invention

This invention relates to devices to treat aneurysms.

### Introduction

An aneurysm is an abnormal bulging or ballooning of the wall of a blood vessel. This results in a blood-filled bulge or sac that extends outwards from the parent blood vessel. Among the causes of aneurysms are disease, trauma, and weakening of the vessel wall. If an aneurysm grows larger, then its walls generally become thinner and weaker. This decrease in wall integrity increases the risk of the aneurysm hemorrhaging blood into the surrounding tissue through a leak or a complete rupture, with serious and possibly fatal health outcomes. Saccular aneurysms that have a relatively-narrow base connection to the parent vessel (also called “narrow-neck aneurysms”) have a greater risk of rupture than fusiform aneurysms (also called “wide-neck aneurysms”).

Cerebral aneurysms, also called “brain aneurysms” or “intracranial aneurysms”, are aneurysms that occur in the intercerebral arteries that supply blood to the brain. One place where cerebral aneurysms often form is a junction of arteries at the base of the brain that is known as the Circle of Willis, where arteries come together and from which these arteries send branches to different areas of the brain. The fluid dynamics of blood flow through this junction put relatively-high pressure on certain places along the vessel walls. Weak spots form in the vessel walls where the arteries come together in this circle.

The prevalence of cerebral aneurysms is estimated at 1-5% of the general population. Some aneurysms are asymptomatic and are never detected. However, some cerebral aneurysms rupture, resulting in a high risk of stroke, disability, and death. Approximately one-third to one-half of people who suffer a ruptured cerebral aneurysm will die within one month of the rupture. Sadly, even among those who survive, approximately one-half suffer significant and permanent deterioration of brain function.

Cerebral aneurysms that rupture, or that are detected and determined to have a high risk of potential rupture, are often treated by clamping a small aneurysm clip across the neck of the aneurysm. The clip stops blood flow through the aneurysm. When blood flow through the aneurysm stops, the blood within the aneurysm starts to embolize and gradually wall tissue begins to form between the parent blood vessel and the aneurysm neck. This helps to restore the wall integrity of the parent blood vessel.

Aneurysm clips often have a pair of clamping arms that are connected by a flexible, spring-action loop. These arms are often opened by compressing the loop. Once open, the arms are positioned on either side of the aneurysm neck and then closed around the neck when compression of the loop is released. Since the locations in which cerebral aneurysms form are often in narrow spaces that are difficult to reach, cerebral aneurysm clips are designed to fit through a narrow access pathway. Similar blood vessel clips or clamps are often used to compress blood vessels in other locations in the body for treatment of non-cerebral aneurysms or for other conditions that require reduction of blood flow through blood vessels or structures.

The subarachnoid space is between the arachnoid membrane and the pia mater surrounding the brain. When the rupture of a cerebral aneurysm releases blood into this space, this event is called a Subarachnoid Hemorrhage or “SAH”. An SAH is a serious health hazard. Approximately one-third to one-half of people with an SAH die within one month and, of those who survive, approximately one-half suffer significant loss of brain function and require help with the activities of daily living afterwards.

The overall chain of events from a ruptured cerebral aneurysm to adverse health outcomes is as follows. First, blood escaping from a ruptured cerebral aneurysm can spill out into the subarachnoid space (an event called a “subarachnoid hemorrhage” or SAH). Second, the results of biodegrading blood products from the escaped blood can cause spasm and contraction of the smooth muscle cells in the walls of cerebral blood vessels (a condition called “cerebral vasospasm”). Third, the contraction of these blood vessel walls can restrict blood flow to downstream tissue, depriving that tissue of oxygen (a condition called “Delayed Ischemic Neurological Deficit” or “DIND”). Fourth, progressive DIND can result in extensive brain tissue necrosis and death.

Implantation of an aneurysm clip onto the neck of a cerebral aneurysm helps to prevent or stop the first step of this chain of events. A clip can prevent or stop an aneurysm from bleeding. However, especially when an aneurysm has already ruptured and blood has already been released into the subarachnoid space, it is vital to also block this chain of events further along the causal chain. Towards this end, prevention and treatment of vasospasm is also an important part of reducing the risk of brain tissue necrosis, stroke, and death associated with cerebral aneurysms.

Calcium antagonists can reduce vasospasm by reducing the amount of calcium that enters the smooth muscle cells of the vessel walls in order to reduce ischemia and risk of stroke. Magnesium, administered in compounds such as anhydrous magnesium, is a calcium antagonist and vasodilator. Some methods of magnesium application, such as topical application to the exterior walls of the affected blood vessels, may be useful for reducing vasospasm and protecting neurons from damage after SAH.

### REVIEW AND LIMITATIONS OF THE PRIOR ART

Before reviewing the prior art concerning the use of clips to treat cerebral aneurysms, we will briefly review the historical development of stents to treat stenosis. This brief review of stents provides a useful historical perspective and analogy for the review of aneurysm clips that follows. Stenosis is a condition wherein the walls of a blood vessel grow thick with plaque. Stenosis can clog the central lumen of the vessel and reduce blood flow. Endovascular stents are medical devices that are expanded within a blood vessel that is affected by stenosis in order to push back the vessel walls, reopen the vessel, and improve blood flow.

For many years, bare metal stents, without any drug coating, were used. However, restenosis often occurred with these bare metal stents. Plaque growth often resumed on the walls of the stents and the central lumen of the vessel often became clogged again. To reduce restenosis, drug-eluting stents were invented. The walls of drug-eluting stents have a coating that contains a drug that gradually seeps out and prevents the growth that would cause restenosis. Although the drugs used in such coatings had existed for years before drug-eluting stents were invented and although bare-metal stents had existed for years before drug-eluting stents were invented, the combination of drug and stent that comprised a drug-eluting stent was deemed, in the patent process, to be innovative, non-obvious, and useful.

This present invention relates to treatment of cerebral aneurysms, not stenosis, but the above background is useful as an introduction to the innovative, non-obvious, and useful invention disclosed herein. A cerebral aneurysm is a condition wherein a blood vessel in the brain bulges or even hemorrhages. Aneurysm clips are medical devices that are applied to the aneurysm from outside the blood vessel in order to stop blood from flowing through the aneurysm.

For several decades, bare metal aneurysm clips (without any drug coating) have been used to treat aneurysms. However, the blood that hemorrhages from a cerebral aneurysm can cause vasospasm and stroke in the brain. Clips do nothing to address blood that has already leaked out. To reduce the chances of vasospasm and stroke after blood has leaked out from an aneurysm, calcium channel antagonists are sometimes administered to the patient.

Nimodipine is one such calcium antagonist. Surgical insertion of nimodipine (in solid, gel, or semi-gel form) into predetermined locations that are a certain distance (eg. from about 5-10 mm) away from cerebral arteries is known in the prior art. However, many cerebral aneurysms are located in areas that are deep within the brain and hard to reach. There are only narrow access pathways through which the neurosurgeon can reach the aneurysm to deploy an aneurysm clip. Also, a neurosurgeon is often faced with significant constraints in terms of surgical time. There may be a sufficient access pathway through the brain tissue and sufficient surgical time available to deploy an aneurysm clip, but not for also implanting multiple boluses of nimodipine in predetermined locations throughout the surrounding brain tissue. Further, although additional empirical research is required to be certain, there may be clinical or biochemical conditions under which treatment with magnesium-based compounds is safer, and more effective, for suppressing vasospasm in the brain than treatment with nimodipine-related compounds.

Local perfusion with calcium antagonists such as Nimodipine during surgery is known in the prior art. However, local perfusion in the area surrounding the blood vessel is limited to the time period during surgery when tissue is pushed aside and the aneurysm is accessible. Local perfusion with calcium antagonists during surgery may not be able to sustain long-term therapeutic levels of calcium antagonists throughout the entire window of vulnerability.

Intravenous and oral administration of calcium antagonists to reduce vasospasm are also known in the prior art. However, intravenous or oral administration may not be as effective as topical administration to the exterior walls of the affected blood vessels because of difficulties crossing the blood-brain barrier.

The prior art includes surgical clips which are applied from outside a blood vessel to stop the vessel from hemorrhaging. Some of these clips may include drug coatings or reservoirs. This art includes: U.S. Patent Application 20090222026 (Rothstein et al., “Surgical Fastening Clips, Systems and Methods for Proximating Tissue”); and U.S. Pat. No. 6,869,436 (Wendlandt, “Surgical Clip with a Self-Releasing Fluid Reservoir”). However this prior art does not teach the use of calcium-antagonizing compounds for treating cerebral aneurysms.

The prior art also includes the separate application of compositions, including calcium-antagonizing compounds, to treat vasospasm. This art includes: U.S. Patent Applications 20060217340 (Braydon et al., “Methods and Products for Treating Hypertension by Modulation of TRPC3 Channel Activity”), 20080305147 (Macdonald, “Drug Delivery System for the Prevention of Cerebral Vasospasm”), 20100035837 (Sasaki, “Therapeutic or Prophylactic Agent for Vasoconstriction”), and 20100092467 (Isenberg, “Prevention of Tissue Ischemia Related Methods and Compositions”); U.S. Pat. No. 6,796,966 (Thomas, “Apparatus and Kits for Preventing of Alleviating Vasoconstriction or Vasospasm in a Mammal”); and PCTs WO/2006/084005 (Wellman et al., “Emergence of a R-Type Ca2+Channel(Cav2.3) Contributes To Cerebral Artery Constriction Following Subarachnoid Hemorrhage”) and WO/2008/154585 (Macdonald et al., “Drug Delivery System for the Prevention of Cerebral Vasospasm”). However this prior art does not teach the use of clips in general, or calcium-antagonist-eluting clips in particular, for treating cerebral aneurysms.

The prior art also includes endovascular stents, deployed inside blood vessels, to provide support for blood vessels. This art includes: U.S. Patent Applications 20100331966 (Borck, “Biocorrodible Implant Having an Active Coating”), 20100106243 (Wittchow, “Implant Made of Biocorrodible Iron or Magnesium Alloy”), 20080262589 (Nagura, “Intravascular Implant”); 20080243234 (Wilcox, “Magnesium Alloy Stent”), and 20070135908 (Zhao, “Absorbable Stent Comprising Coating for Controlling Degradation and Maintaining Ph Neutrality”); and U.S. Pat. No. 7,452,373 (Kaul et al., “Stent Coated with Magnesium-Based Compound for Reducing Thrombosis”). However this prior art does not teach the use of clips outside a blood vessel in general, or calcium-antagonist-eluting clips in particular, for treating cerebral aneurysms.

### SUMMARY AND ADVANTAGES OF THIS INVENTION

This invention is an implantable device that is attached to the exterior of an aneurysm, or to the exterior of some other portion of a blood vessel wall, wherein this device elutes, or otherwise emits, an emitted substance to prevent or treat vasospasm. This device is better than a bare metal aneurysm clip alone because bare metal clips offer no biochemical protection against vasospasm and stroke due to hemorrhaged blood. This calcium-antagonist-eluting clip is also better than use of a bare mental aneurysm clip with separate administration of a calcium antagonizing compound because: it allows ongoing therapeutic elution of the compound within the brain long after the surgery is over; it saves time compared to separate clip implantation and compound administration; and it reduces the risks of dosage error from compound administration that is separate from clip implantation. Based on a review of the prior art, the prior art does not appear to teach or suggest a calcium-antagonist-eluting aneurysm clip. For these reasons, a calcium-antagonist-eluting aneurysm clip appears to be an innovative, non-obvious, and useful invention.

This invention may be embodied as a magnesium-eluting aneurysm clip. A magnesium-eluting aneurysm clip provides “two-for-one” blockage of two links in the above-described four-link chain from cerebral aneurysm to ischemia and stroke. Without requiring a larger tissue opening or additional surgical time (since the neurosurgeon is already deploying a clip), a magnesium-eluting aneurysm clip not only stops blood from escaping out of the aneurysm, but it also helps to prevent vasospasm from any blood that has already escaped. The neurosurgeon can achieve long-term therapeutic levels of topical administration of magnesium on the exterior of the affected vessels without the risk of a larger opening or longer operation. This is a considerable advantage over traditional aneurysm clips, over intravenous or oral administration of calcium antagonists, over short-term topical administration of calcium antagonists, and over time-consuming insertion of multiple boluses of nimodipine in different locations throughout the surrounding brain tissue.

This invention may also be embodied as a flexible wrap (or sheath, cord, filament, suture, fabric, or balloon) that is attached to the exterior of a blood vessel by partially or fully encircling the device around the vessel wall. Such a wrap embodiment may be used in combination with the aneurysm clip embodiment of this invention described above. This flexible wrap embodiment does require additional surgical time for implantation as compared to deployment of a magnesium-eluting aneurysm clip alone. However, such a wrap may provide advantageous additional levels of magnesium through direct contact with the parent vessel. Such flexible attachment of a magnesium-eluting device to the exterior of a vessel does not appear to be found in the prior art. In an example, this flexible wrap embodiment may be bioabsorbable over the span of the critical one-month window of vulnerability for vasospasm.

### DETAILED DESCRIPTION OF THE FIGURES

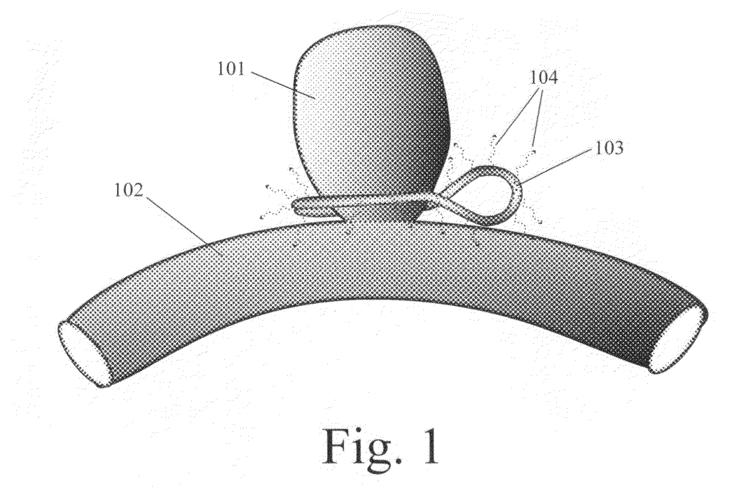

FIG. 1 shows one possible embodiment of this invention. FIG. 1 shows a side view of an aneurysm 101 ballooning out from a parent blood vessel 102. In FIG. 1, the ends of parent vessel 102 are shown as having been conceptually truncated (cross-sectionally cut) in order to highlight the tubular nature of the vessel and constrain it to the page size. In actuality, the ends of parent vessel 102 would continue past the side boundaries of the page.

FIG. 1 shows an embodiment of this invention in which an aneurysm clip 103 that is eluting magnesium 104 has been deployed on the base connection (the “neck”) of aneurysm 101 where it attaches to parent vessel 102. Not only do the mechanical properties of this clip stop blood flow through the aneurysm, but the biochemical properties of this clip reduce vasospasm from any blood that escaped from the aneurysm before the clip was deployed. An aneurysm with a relatively generic design is shown because the exact design of the aneurysm clip is not central to the innovation of this invention.

In this embodiment, magnesium is eluted from the clip in the form of anhydrous magnesium. In another examples, magnesium may be eluted, or otherwise emitted, from the aneurysm clip in other formulations. In this embodiment, magnesium is eluted from a coating on the aneurysm clip. Several methods of coating implantable devices with substance-eluting coatings are known in the prior art and the exact method is not central to this invention. In other examples, magnesium may be eluted, or otherwise emitted, from: an alloy used in the formation of the device; nanoscale substance-containing structures in, or on, the device; microscale substance-containing structures in, or on, the device; or one or more millimeter-scale substance-containing structures in the device.

In this example, the substance that is eluted is a magnesium-based compound. In another examples, the substance that is eluted may be nimodipine or some other substance that blocks, inhibits, and/or antagonizes calcium and/or the calcium channels. In this example, the core of the aneurysm clip is made from titanium. In other examples, the device may be made from one or more materials selected from the group consisting of: alumina fiber, carbon fiber, cobalt alloy, Co—Cr alloy, epoxy resin, fluorine resin, magnesium alloy, methacrylic resin, polyether sulfone, polyethylene, polymethyl methacrylate, polypropylene, polystyrene, polytetrafluoroethylene, sialon, SiC fiber, silicon carbide, silicon nitride, silicone resin, stainless steel, titanium alloy, and unsaturated polyester resin.

In an example, the rate of emission of magnesium from this device may be altered remotely, from outside the person's body, after implantation by: electromagnetic communication with a Micro Electronic Mechanical System (MEMS) component that is part of, or connected to, the device; electromagnetic communication with a millimeter-scale electronic component that is part of, or connected to, the device; or the effect of electromagnetic radiation on a substance-emitting coating, layer, or structure that is part of the device.

In this example, the implantable device is an aneurysm clip. In another example, the device may be a surgical clip or clamp that is attached to the exterior of some other portion of a blood vessel wall in order to address vasospasm in a condition other than an aneurysm.

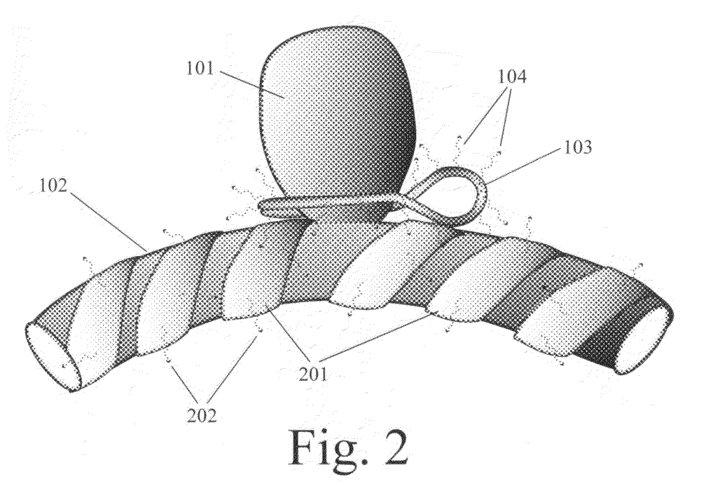

FIG. 2 shows a second embodiment of this invention. In this example, this second embodiment is used in combination with the first embodiment (the magnesium-eluting aneurysm clip) that was shown in FIG. 1. FIG. 2 shows a second embodiment of this invention comprising a magnesium-eluting flexible spiral wrap 201 that is attached to the exterior of parent vessel 102 by gently encircling the wrap 201 around parent vessel 102. In FIG. 2, magnesium 202 is conceptually shown (by wavy dotted arrows) as eluting from the wrap, directly exposing the wall of the parent vessel 102 to magnesium. In this example, spiral wrap 201 is bioabsorbable.

In other examples of this embodiment, the magnesium-eluting, vasospasm-reducing device may be a sheath, cord, filament, suture, fabric, balloon or other flexible member that is attached to the exterior of some portion of a blood vessel wall by partially or fully encircling the device around the blood vessel wall. In another examples, the device may encircle the aneurysm itself. In another example, a flexible magnesium-eluting device may be attached to an aneurysm, or to the exterior of some other portion of a blood vessel wall, by means of adhesion or fibrosis.

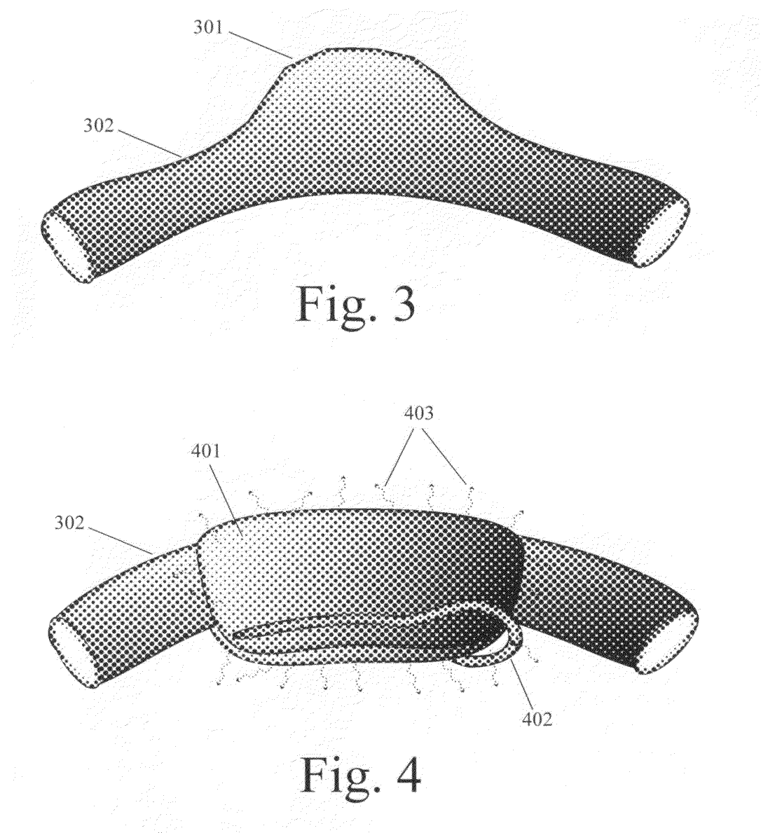

FIGS. 3 and 4 show another example of a magnesium-eluting wrap embodiment of this invention being used in combination with a magnesium-eluting aneurysm clip embodiment of this invention. FIG. 3 shows a fusiform or “wide-neck” aneurysm 301 that is bulging from the top portion of its parent vessel 302. A wide-neck aneurysm such as this can be difficult to clip.

FIG. 4 shows treatment of this wide-neck aneurysm by deployment of a magnesium-eluting wrap 401. In FIG. 4, magnesium-eluting wrap 401 has been snugly wrapped around the circumference of the aneurysm 301 and the parent vessel 302. The two ends of wrap 401 are held tightly together by the compression of magnesium-eluting clip 402. In FIG. 4, the wide-neck aneurysm is no longer visible because it is compressed and covered by wrap 401. In another variation on this example in FIG. 4, the ends of magnesium-eluting wrap 401 may be held together by adhesion, by fibrosis, by suturing, or by stapling. In this example, wrap 401 is not bioabsorbable because it provides useful structural support for the weakened portion of the parent vessel wall.

In FIG. 4, as in previous figures, magnesium 403 is conceptually shown (by wavy dotted arrows) as eluting from the wrap. This directly exposes the wall of the parent vessel 302 to magnesium. In this manner, not only does wrap 401 provide structural support to constrain bulging wide-neck aneurysm 301, but it also provides biochemical means to reduce the risk of vasospasm from any blood that has leaked, or may leak, out from the aneurysm.

We claim:

1. An implantable device that is attached to the exterior of an aneurysm, or to the exterior of some other portion of a blood vessel wall, wherein this device elutes, or otherwise emits, an emitted substance to prevent or treat vasospasm.

2. The implantable device in claim 1 wherein this device is a clip, clamp, or other compressive device that is attached to the exterior of an aneurysm, or to the exterior of some other portion of a blood vessel wall, by compression.

3. The implantable device in claim 1 wherein this device is a wrap, sheath, cord, filament, suture, fabric, balloon or other flexible member that is attached to the exterior of an aneurysm, or to the exterior of some other portion of a blood vessel wall, by partially or fully encircling the device around the aneurysm or other portion of a blood vessel wall.

4. The implantable device in claim 1 wherein this device is attached to the exterior of an aneurysm, or to the exterior of some other portion of a blood vessel wall, by means of adhesion or fibrosis.

5. The implantable device in claim 1 wherein the emitted substance that is eluted, or otherwise emitted, from one or more sources within, or on, the device is selected from the group consisting of: a coating on the device; a layer of the device; an alloy used in the formation of the device; nanoscale substance-containing structures in, or on, the device; microscale substance-containing structures in, or on, the device; and one or more millimeter-scale substance-containing structures in the device.

6. The implantable device in claim 1 wherein the emitted substance blocks, inhibits, and/or antagonizes calcium and/or the calcium channels.

7. The implantable device in claim 1 wherein the emitted substance is, or contains, magnesium or a magnesium-containing compound, such as anhydrous magnesium.

8. The implantable device in claim 1 wherein this device is used to prevent or treat vasospasm in a condition other than an aneurysm.

9. The implantable device in claim 1 wherein this device is made from one or more materials selected from the group consisting of: alumina fiber, carbon fiber, cobalt alloy, Co—Cr alloy, epoxy resin, fluorine resin, magnesium alloy, methacrylic resin, polyether sulfone, polyethylene, polymethyl methacrylate, polypropylene, polystyrene, polytetrafluoroethylene, sialon, SiC fiber, silicon carbide, silicon nitride, silicone resin, stainless steel, titanium or titanium alloy, and unsaturated polyester resin.

10. The implantable device in claim 1 wherein the rate of emission of the emitted substance from the device can be altered remotely, from outside the person's body, after implantation by: electromagnetic communication with a Micro Electronic Mechanical System (MEMS) component that is part of, or connected to, the device; electromagnetic communication with a millimeter-scale electronic component that is part of, or connected to, the device; or the effect of electromagnetic radiation on a substance-emitting coating, layer, or structure that is part of the device.

11. The implantable device in claim 1 wherein the device is biodegradable and/or bioabsorbable.

12. An implantable device that is attached to the exterior of an aneurysm, or to the exterior of some other portion of a blood vessel wall,
wherein this device elutes, or otherwise emits, an emitted substance to prevent or treat vasospasm;
wherein this emitted substance blocks, inhibits, and/or antagonizes calcium and/or the calcium channels; and
wherein this device is selected from the group consisting of: a clip, clamp, or other compressive device that is attached to the exterior of an aneurysm, or to the exterior of some other portion of a blood vessel wall, by compression; a wrap, sheath, cord, filament, suture, fabric, balloon or other flexible member that is attached to the exterior of an aneurysm, or to the exterior of some other portion of a blood vessel wall, by partially or fully encircling the device around the aneurysm or other portion of a blood vessel wall; or a device is attached to the exterior of an aneurysm, or to the exterior of some other portion of a blood vessel wall, by means of adhesion or fibrosis.

13. The implantable device in claim 12 wherein the emitted substance that is eluted, or otherwise emitted, from one or more sources within, or on, the device is selected from the group consisting of: a coating on the device; a layer of the device; an alloy used in the formation of the device; nanoscale substance-containing structures in, or on, the device; microscale substance-containing structures in, or on, the device; and one or more millimeter-scale substance-containing structures in the device.

14. The implantable device in claim 12 wherein the emitted substance is, or contains, magnesium or a magnesium-containing compound, such as anhydrous magnesium.

15. The implantable device in claim 12 wherein this device is used to prevent or treat vasospasm in a condition other than an aneurysm.

16. The implantable device in claim 12 wherein this device is made from one or more materials selected from the group consisting of: alumina fiber, carbon fiber, cobalt alloy, Co—Cr alloy, epoxy resin, fluorine resin, magnesium alloy, methacrylic resin, polyether sulfone, polyethylene, polymethyl methacrylate, polypropylene, polystyrene, polytetrafluoroethylene, sialon, SiC fiber, silicon carbide, silicon nitride, silicone resin, stainless steel, titanium or titanium alloy, and unsaturated polyester resin.

17. The implantable device in claim 12 wherein the rate of emission of the emitted substance from the device can be altered remotely, from outside the person's body, after implantation by: electromagnetic communication with a Micro Electronic Mechanical System (MEMS) component that is part of, or connected to, the device; electromagnetic communication with a millimeter-scale electronic component that is part of, or connected to, the device; or the effect of electromagnetic radiation on a substance-emitting coating, layer, or structure that is part of the device.

18. The implantable device in claim 12 wherein the device is biodegradable and/or bioabsorbable.

19. An implantable device that is attached to the exterior of an aneurysm, or to the exterior of some other portion of a blood vessel wall,
wherein this device elutes, or otherwise emits, an emitted substance to prevent or treat vasospasm;
wherein the emitted substance is, or contains, magnesium or a magnesium-containing compound, such as anhydrous magnesium; and
wherein this device is selected from the group consisting of: a clip, clamp, or other compressive device that is attached to the exterior of an aneurysm, or to the exterior of some other portion of a blood vessel wall, by compression; a wrap, sheath, cord, filament, suture, fabric, balloon or other flexible member that is attached to the exterior of an aneurysm, or to the exterior of some other portion of a blood vessel wall, by partially or fully encircling the device around the aneurysm or other portion of a blood vessel wall; or a device is attached to the exterior of an aneurysm, or to the exterior of some other portion of a blood vessel wall, by means of adhesion or fibrosis.

20. The implantable device in claim 19 wherein the emitted substance that is eluted, or otherwise emitted, from one or more sources within, or on, the device is selected from the group consisting of: a coating on the device; a layer of the device; an alloy used in the formation of the device; nanoscale substance-containing structures in, or on, the device; microscale substance-containing structures in, or on, the device; and one or more millimeter-scale substance-containing structures in the device.
